(19) United States
(12) Patent Application Publication
(10) Pub. No.: US 2012/0209322 A1
(43) Pub. Date: Aug. 16, 2012

(54) TACKING DEVICE

(75) Inventor: Vihar C. Surti, Winston-Salem, NC (US)

(73) Assignee: Wilson-Cook Medical Inc., Winston-Salem, NC (US)

(21) Appl. No.: 13/454,618

(22) Filed: Apr. 24, 2012

Related U.S. Application Data
(62) Division of application No. 12/428,226, filed on Apr. 22, 2009.
(60) Provisional application No. 61/047,293, filed on Apr. 23, 2008.

(51) Int. Cl. A61B 17/08

(52) U.S. Cl. 606/213

(57) ABSTRACT

The present embodiments provide apparatus and methods suitable for coupling a graft member to tissue, closing a bodily opening, and the like. At least one proximal deployable member and at least one distal deployable member are provided, each having a contracted state suitable for delivery and further comprising an expanded state. In the expanded state, the proximal and distal deployable members are configured to secure the graft member to the tissue. Optionally, a loop member may be provided for receiving a suture for further securing the graft member to the tissue.

### PRIORITY CLAIM

This invention claims the benefit of priority of U.S. Provisional Application Ser. No. 61/047,293, entitled “Tacking Device,” filed Apr. 23, 2008, the disclosure of which is hereby incorporated by reference in its entirety.

### BACKGROUND

The present embodiments relate generally to medical devices, and more particularly, to apparatus and methods for coupling a graft member to tissue, closing a bodily opening, and the like.

Perforations in tissue or bodily walls may be formed intentionally or unintentionally. For example, an unintentional ventral abdominal hernia may be formed in the abdominal wall due to heavy lifting, coughing, strain imposed during a bowel movement or urination, fluid in the abdominal cavity, or other reasons.

Intentional perforations may be formed, for example, during surgical procedures such as translumenal procedures. In a translumenal procedure, one or more instruments, such as an endoscope, may be inserted through a visceral wall, such as the stomach wall. During a translumenal procedure, a closure instrument may be used to close the perforation in the visceral wall. Depending on the structure comprising the perforation, it may be difficult to adequately close the perforation and prevent leakage of bodily fluids.

Attempts to seal perforations have been attempted by coupling a graft member to tissue. For example, during hernia repair, a graft material such as a mesh or patch may be disposed to cover the perforation. The graft material may completely overlap with the perforation, and the edges of the graft material may at least partially overlap with tissue surrounding the perforation. The graft material then may be secured to the surrounding tissue in an attempt to effectively cover and seal the perforation.

In order to secure the graft material to the surrounding tissue, sutures commonly are manually threaded through the full thickness of the surrounding tissue. In the case of a ventral abdominal hernia, the sutures may be threaded through the thickness of the abdominal wall, then tied down and knotted. However, such manual suturing techniques may be time consuming and/or difficult to perform.

In addition to covering and sealing perforations, there are various other instances in which it may be desirable to couple a graft material to tissue. For example, it may become necessary or desirable to couple the graft material to a region of tissue for purposes of reconstructing the local tissue. Whether a graft material is coupled to tissue to reconstruct local tissue, seal a perforation, or another purpose, it would be desirable to provide apparatus and methods for quickly and effectively coupling the graft material to the tissue.

### SUMMARY

The present embodiments provide a tacking device for engaging tissue, which may be useful for coupling a graft to tissue or facilitating closure of a bodily opening. In one embodiment, a tacking device is provided comprising a first wire having proximal and distal ends, a proximal deployable member formed at the proximal end of the first wire, and a distal deployable member formed at the distal end of the first wire. Similarly, a second wire has proximal and distal ends, a proximal deployable member formed at the proximal end of the second wire, and a distal deployable member formed at the distal end of the second wire. Further, a third wire has proximal and distal ends, a proximal deployable member formed at the proximal end of the third wire, and a distal deployable member formed at the distal end of the third wire. Each of the proximal deployable members of the first, second and third wires has contracted and expanded states, and further, each of the distal deployable members of the first, second and third wires has contracted and expanded states. Central regions of each of the first, second and third wires are affixed to one another.

In one embodiment, each of the proximal and distal deployable members of the first, second and third wires comprise hook-shaped configurations in the expanded states. The proximal and distal deployable members each may comprise a nickel-titanium alloy that is configured to self-expand to the hook-shaped configuration.

In one embodiment, an end region of the distal deployable member of the first wire is substantially parallel to a longitudinal axis of the first wire in the contracted state, and further, the distal deployable member of the first wire retroflexes in the expanded state to be radially spaced apart from, and substantially parallel to, the longitudinal axis of the first wire. Similarly, an end region of the proximal deployable member of the first wire may be substantially parallel to a longitudinal axis of the first wire in the contracted state, and retroflexes in the expanded state to be radially spaced apart from, and substantially parallel to, the longitudinal axis of the first wire.

The tacking device may be delivered to a target site using an insertion tool comprising a hollow lumen having an inner diameter configured to receive the tacking device having the proximal and distal deployable members. The proximal and distal deployable members are configured to be held in the contracted states when disposed within the hollow lumen. In the contracted states, the proximal and distal deployable members may be oriented in substantially longitudinally directions with respect to the insertion tool.

In an exemplary use of the tacking device for coupling graft member to tissue, the graft member may be positioned over a selected region of the tissue. The insertion tool may be advanced to penetrate through the graft member and through a portion of the tissue. The insertion tool then may be proximally retracted with respect to the tacking device to cause the distal deployable members to expand and engage the tissue. Further retraction of the insertion tool with respect to the tacking device may cause the proximal deployable members to expand and engage the graft member. A stylet loaded into the hollow lumen may abut the proximal deployable members to facilitate retraction of the insertion tool with respect to the tacking device. If desired, multiple tacking devices may be sequentially loaded within the hollow lumen of the insertion tool and then sequentially deployed to secure the tissue to the graft material at multiple different locations.

Optionally, at least one loop member configured to receive a suture may be used for further securing the graft member to the tissue. The loop member may be integrally formed with a wire of the tacking device by bending a portion of the wire in an arch-shaped manner. In use, multiple tacking devices comprising loop members may be deployed, and a suture may be threaded through the loop members and actuated in a purse-string fashion.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a perspective view of a tacking device.

FIG. 2 is a perspective view of a distal region of an insertion tool and the tacking device of FIG. 1.

FIG. 3 is a perspective, cut-away view illustrating multiple tacking devices in a delivery configuration.

FIG. 4 is a schematic view illustrating a ventral hernia.

FIG. 5 is a schematic view illustrating a graft member used to cover the ventral hernia of FIG. 4.

FIG. 6 is a schematic view of a method step for treating the ventral hernia of FIG. 4.

FIG. 7 is a side-sectional view taken along line A-A of FIG. 6.

FIG. 8 is a side-sectional view showing multiple tacking devices deployed in expanded configurations.

FIG. 9 is a schematic view illustrating multiple deployed tacking devices used to treat the ventral hernia of FIG. 4.

FIG. 10 is a perspective view of an alternative tacking device.

FIG. 11 is a side-sectional view illustrating one method of use of multiple tacking devices of FIG. 10.

FIG. 12 is a perspective view of an alternative tacking device.

FIG. 13 is a side view of a further alternative tacking device.

FIGS. 14-15 are side views of an alternative deployable member of a tacking device.

### DETAILED DESCRIPTION OF THE PREFERRED EMBODIMENTS

In the present application, the term “proximal” refers to a direction that is generally towards a physician during a medical procedure, while the term “distal” refers to a direction that is generally towards a target site within a patient's anatomy during a medical procedure.

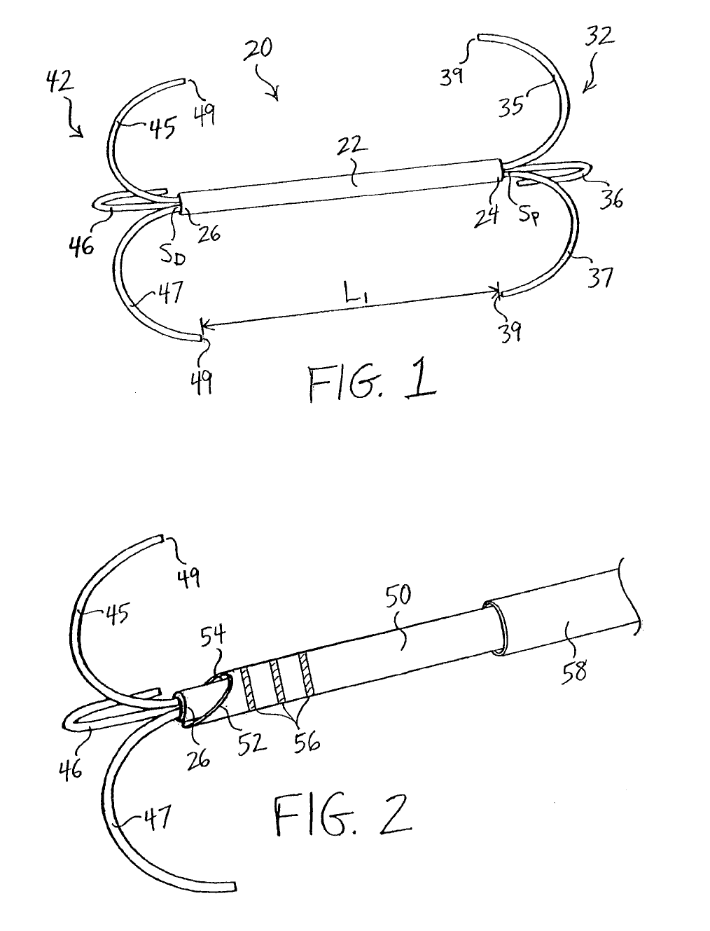

Referring now to FIG. 1, a first embodiment of a tacking device 20 is shown. In this embodiment, the tacking device 20 comprises at least one tube member 22 having a proximal end 24 and a distal end 26. The tacking device 20 further comprises a proximal deployment mechanism 32 and a distal deployment mechanism 42. In the embodiment of FIG. 1, the proximal deployment mechanism 32 comprises three proximal deployable members 35-37, while the distal deployment mechanism 42 comprises three distal deployable members 45-47. The proximal deployable members 35-37 extend proximally from the proximal end 24 of the tube member 22, while the distal deployable members 45-47 extend distally from the distal end 26 of the tube member 22, as shown in FIG. 1. In the embodiment of FIG. 1, since the device is symmetrical, it may be loaded into an insertion tool with either end first, as explained further below.

The proximal deployable members 35-37 and the distal deployable members 45-47 each may be affixed relative to the tube member 22. In one embodiment, each of the proximal and distal deployable members 35-37 and 45-47 may be separate and discrete elements. Accordingly, six separate deployable members may be provided. Specifically, the three proximal deployable members 35-37 may be coupled to the tube member 22 near the proximal end 24 of the tube member 22. The three proximal deployable members 35-37 may be coupled to the proximal end 24 of the tube member 22 using an adhesive, frictional fit, mechanical device or other suitable mechanism or processes. Similarly, the three distal deployable members 45-47 may be coupled to the distal end 26 of the tube member 22 using an adhesive, frictional fit, mechanical device or other suitable mechanism.

In an alternative embodiment, instead of providing six discrete deployable members, three wires may be disposed through the entirety of tube member 22. In this embodiment, a first wire may comprise a proximal end that forms the deployable member 35 and a distal end that forms the deployable member 45, while a central region of the same wire is disposed through the entirety of the tube member 22. Similarly, second and third wires may be disposed through the entirety of the tube member 22 to form the remaining proximal and distal deployable members. In this embodiment, the three wires that extend through the length of the tube member 22 may be affixed to an interior surface of the tube member 22, for example, using an adhesive or mechanical device. The three wires also may be sized to create a frictional fit against each other and/or an interior surface of the tube member 22, thereby inhibiting movement of the proximal and distal deployable members 35-37 and 45-47 in longitudinal directions with respect to the tube member 22.

While six total deployable members 35-37 and 45-47 are depicted, including three at both the proximal and distal ends of the tacking device 20, it will be apparent that greater or fewer deployable members may be employed. Moreover, the deployable members 35-37 and 45-47 may comprise any shape suitable for engaging, penetrating and/or abutting tissue, for purposes explained further below, and need not necessarily assume the expanded shape depicted in FIGS. 1-2.

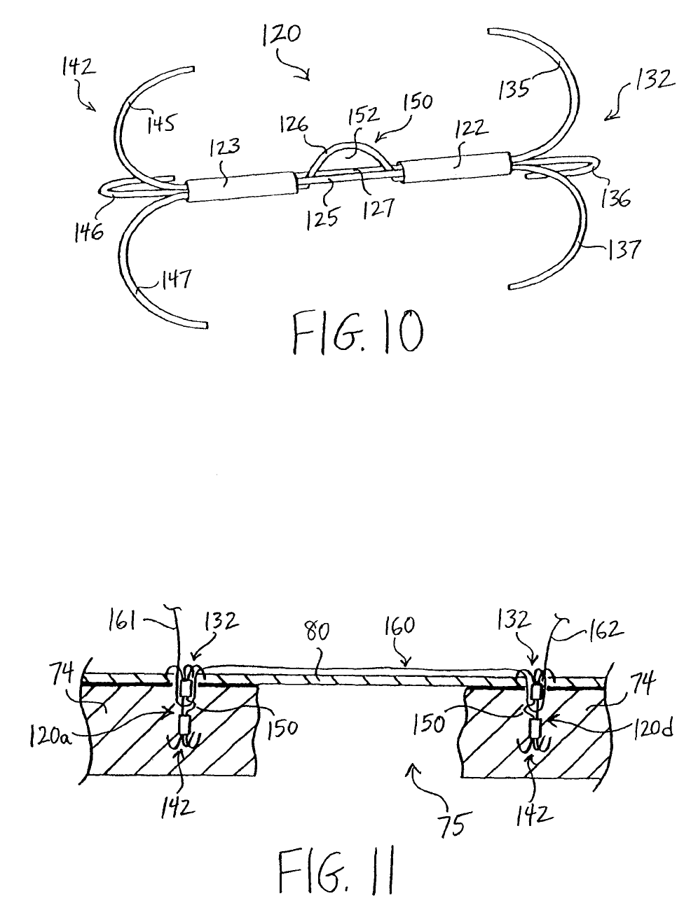

The tube member 22 may comprise any suitable shape and material. Solely by way of example, the tube member 22 may comprise stainless steel or a biocompatible plastic. The tube member 22 may be cylindrically-shaped, as depicted in FIG. 1, which may facilitate insertion through a lumen of an insertion tool 50. Further, the tube member 22 may comprise one solid tube, or alternatively may comprise one or more tubes that may comprise slots, holes, cut-out regions and the like, for example, as shown and explained below with respect to the embodiment of FIGS. 10-11.

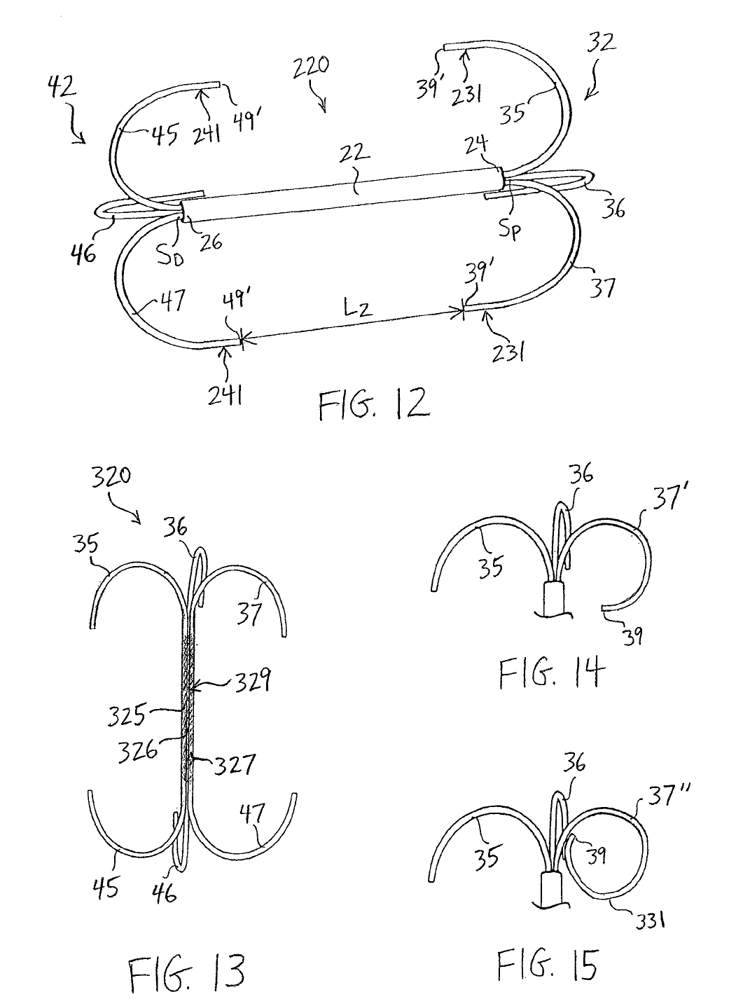

Alternatively, as explained further below with respect to FIGS. 10 and 13, the tube member 22 may be omitted entirely in the case where a first wire integrally forms the proximal and distal deployable members, a second wire integrally forms the proximal and distal deployable members, and a third wire integrally forms the proximal and distal deployable members. In the latter embodiment, central regions of the first, second and third wires may be affixed together, for example, using a solder or weld, to maintain the structural rigidity of the components.

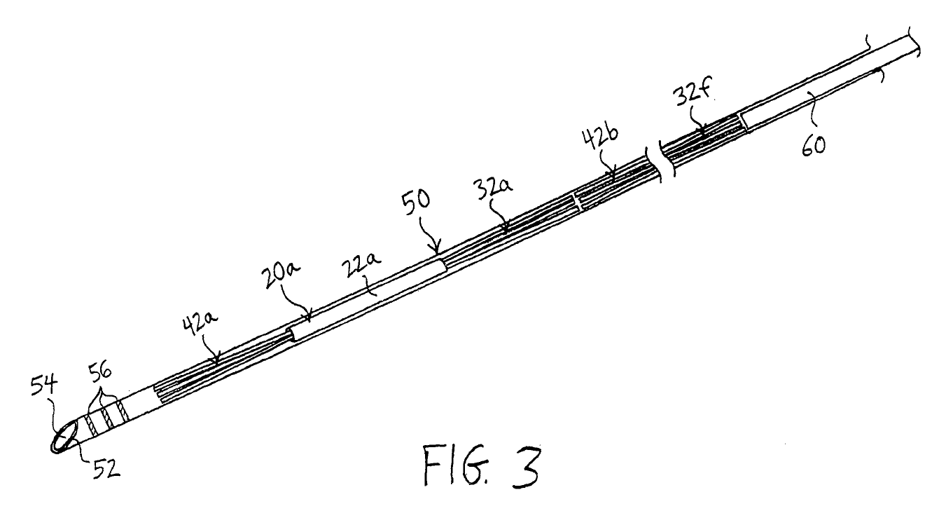

Referring still to FIGS. 1-3, the proximal and distal deployable members 35-37 and 45-47 each comprise a contracted delivery configuration, as shown in FIG. 3 below, and further comprise an expanded deployed configuration, as shown in FIG. 1. In one embodiment, each of the deployable members 35-37 and 45-47 may comprise a hook-shaped configuration in the expanded state. For example, the deployable members 35-37 and 45-47 may comprise a curvature of about 90 to about 360 degrees in the expanded state, and more preferably about 180 degrees, as shown in FIGS. 1-2. Where the deployable members 35-37 and 45-47 “retroflex” and comprises a curvature of about 180 degrees, the ends 39 and 49 of the proximal and distal deployable members are oriented substantially parallel to the tube member 22. Moreover, the ends 39 and 49 may be radially spaced apart from one another in the expanded state, as shown in FIG. 1. In this configuration, the ends 39 and 49 may be well-suited for engaging, grasping, piercing and/or abutting tissue or graft material.

A separation point S_{P} generally defines a point where the proximal deployable members 35-37 begin to radially space apart from one another, while a separation point S_{D} generally defines a point where the distal deployable members 45-47 begin to radially space apart from one another, as shown in FIG. 1. In one embodiment, the ends 39 of the proximal deployable members 35-37 are disposed distal to the separation point Sp, while the ends 49 of the distal deployable members 45-47 are disposed proximal to the separation point S_{D}, as depicted in FIG. 1.

Further, a longitudinal distance L₁ between the ends 39 and 49 of the tacking device 20 may be varied to engage tissue in a desirable manner. For example, the longitudinal distance L₁ may be dimensioned to be substantially equal to or less than the combined thickness t₁ and t₂ of a tissue 74 and a graft member 80, respectively, as shown in FIG. 8 below, thereby providing a desired compressive force upon the tissue 74 and the graft member 80.

The dimension of the tacking device 20 may be tailored based on a particular surgical procedure, a particular patient's anatomy and/or other factors. However, for illustrative purposes, in a ventral hernia repair operation, the longitudinal length of the tube member 22 may range from about 2 mm to about 10 mm, the straightened (delivery or non-curved) length of the proximal deployable members 35-37 may range from about 5 mm to about 50 mm, the straightened (delivery or non-curved) length of the distal deployable members 45-47 may range from about 5 mm to about 50 mm, the longitudinal distance L₁ between the ends 39 and 49 may range from about 5 mm to about 30 mm, the outer diameter of the tube member 22 may range from about 0.3 mm to about 1.5 mm, and the outer diameter of the deployable member 35-37 and 45-47 may range from about 0.1 mm to about 0.5 mm. Such dimensions are provided for reference purposes only and are not intended to be limiting.

The deployable members 35-37 and 45-47 may comprise a shape-memory material, such as a nickel-titanium alloy (nitinol). If a shape-memory material such as nitinol is employed, the deployable members 35-37 and 45-47 may be manufactured such that they can assume the preconfigured expanded state shown in FIG. 1 upon application of a certain cold or hot medium. More specifically, a shape-memory material may undergo a substantially reversible phase transformation that allows it to “remember” and return to a previous shape or configuration. For example, in the case of nitinol, a transformation between an austenitic phase and a martensitic phase may occur by cooling and/or heating (shape memory effect) or by isothermally applying and/or removing stress (superelastic effect). Austenite is characteristically the stronger phase and martensite is the more easily deformable phase.

In an example of the shape-memory effect, a nickel-titanium alloy having an initial configuration in the austenitic phase may be cooled below a transformation temperature (M_{f}) to the martensitic phase and then deformed to a second configuration. Upon heating to another transformation temperature (A_{f}), the material may spontaneously return to its initial, predetermined configuration, as shown in FIG. 1. Generally, the memory effect is one-way, which means that the spontaneous change from one configuration to another occurs only upon heating. However, it is possible to obtain a two-way shape memory effect, in which a shape memory material spontaneously changes shape upon cooling as well as upon heating.

Alternatively, the deployable members 35-37 and 45-47 may be made from other metals and alloys that are biased, such that they may be restrained by the insertion tool 50 prior to deployment, but are inclined to return to their relaxed, expanded configuration upon deployment. Solely by way of example, the deployable members 35-37 and 45-47 may comprise other materials such as stainless steel, cobalt-chrome alloys, amorphous metals, tantalum, platinum, gold and titanium. The deployable members 35-37 and 45-47 also may be made from non-metallic materials, such as thermoplastics and other polymers. As noted above, the deployable members 35-37 and 45-47 may comprise any shape suitable for engaging, penetrating and/or abutting tissue, for purposes explained further below, and need not necessarily assume the curved shape depicted in FIGS. 1-2.

Referring to FIGS. 2-3, one or more tacking devices 20 may be delivered to a target site in a patient's anatomy using an insertion tool 50. In one embodiment, the insertion tool 50 is capable of carrying multiple different tacking devices, such as six tacking devices 20a-20f, as shown in FIG. 9 and described below. In FIG. 3, one complete tacking device 20a is shown in the contracted state, while portions of the distal deployment mechanism 42b of another tacking device 20b, and the proximal deployment mechanism 32f of another tacking device 20f, are also shown.

In one embodiment, the insertion tool 50 comprises a needle-like body having a sharpened distal tip 52 and a hollow lumen 54, as shown in FIGS. 2-3. The insertion tool 50 may be manufactured from stainless steel or any other suitable material, and may comprise an endoscopic ultrasound (EUS), or echogenic, needle. Solely by way of example, the insertion tool 50 may comprise the EchoTip® Ultrasound Needle, or the EchoTip® Ultra Endoscopic Ultrasound Needle, both manufactured by Cook Endoscopy of Winston-Salem, N.C.

The hollow lumen 54 of the insertion tool 50 may comprise an inner diameter than is larger than an outer diameter of the tacking device 20. Therefore, one or more tacking devices, such as six tacking devices 20a-20f, may be loaded into the hollow lumen 54 in a delivery configuration, as shown in FIG. 3. In the delivery configuration, the proximal and distal deployable members 35-37 and 45-47 of each tacking device 20a-20f may comprise a substantially longitudinally-oriented profile, i.e., oriented along a longitudinal axis of the insertion tool 50.

The multiple tacking devices 20a-20f may be inserted into the hollow lumen 54 of the insertion tool 50 in a sequential manner, whereby the proximal deployment mechanism 32a of the first tacking device 20a may abut the distal deployment mechanism 42b of the second tacking device 20b, as depicted in FIG. 3. The distal deployment mechanism 42a of the first tacking device 20a may be loaded a distance away from the sharpened distal tip 52 of the insertion tool 50 to prevent inadvertent deployment.

A stylet 60 may be disposed for longitudinal movement within the hollow lumen 52 of the insertion tool 50, as shown in FIG. 3. The stylet 60 may comprise stainless steel or any other suitable material. The stylet 60 is disposed proximal to the proximal deployment mechanism 32f of the final sequential tacking device 20f, as shown in FIG. 3. During use, the insertion tool 50 may be proximally retracted, while the stylet 60 may be held longitudinally steady, to facilitate sequential deployment of each of the tacking devices 20a-20f, as explained further below.

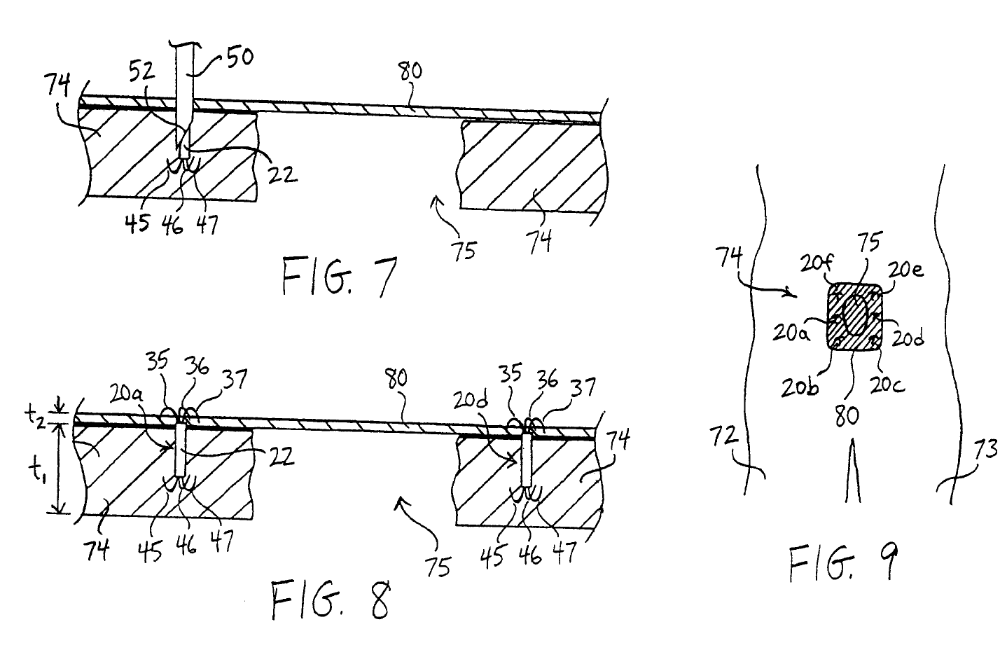

The insertion tool 50 may comprise one or more markers 56, as shown in FIGS. 2-3, which may be disposed near the distal end of the insertion tool 50. The markers 56 may be configured to be visualized under fluoroscopy of other imaging techniques to facilitate location of the distal end of the insertion tool, for example, so that a physician may determine how far the insertion tool 50 has penetrated into tissue 74, as depicted in FIGS. 7-8. Optionally, a sheath member 58 having an inner diameter larger than an outer diameter of the insertion tool 50, as shown in FIG. 2, may be longitudinally advanced over the insertion tool 50, for various purposes explained further below. As will be explained further below, the insertion tool 50 may be used in conjunction with another device, such as an endoscope, and may be delivered through a working lumen of an endoscope or similar device.

Referring now to FIGS. 4-9, one or more tacking devices 20 described above may be used to facilitate treatment of a perforation 75 using a graft member 80. In the example shown, the perforation 75 is a ventral hernia located in the abdominal wall 74. The right and left legs 72 and 73 of a patient 70 are shown for illustrative purposes. While treatment of a ventral hernia is shown for illustrative purposes, it will be apparent that the tacking devices described herein may be used in a wide range of medical procedures, including but not limited to any exemplary procedures described herein.

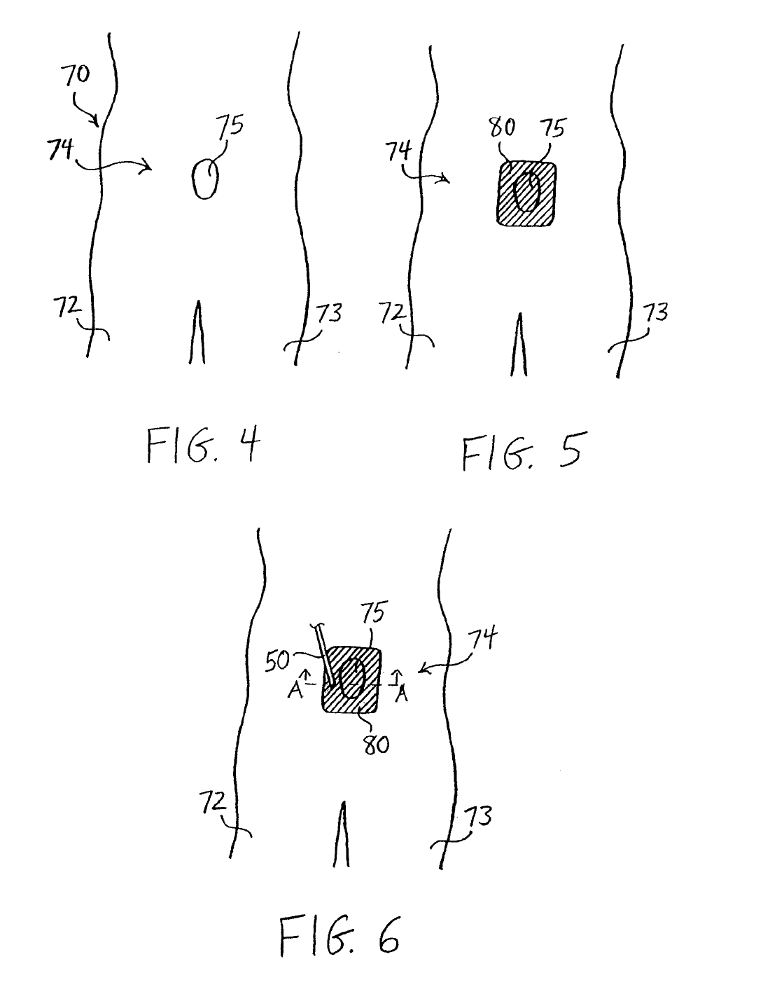

The initial stages of the ventral hernia repair may be performed using techniques that are known. Specifically, an open technique or laparoscopic technique may be employed. In an open technique, an incision may be made in the abdominal wall and fat and scar tissue may be removed from the area. A graft member 80 then may be applied so that it overlaps the perforation 75, preferably by several millimeters or centimeters in each direction, as depicted in FIG. 5. In a laparoscopic technique, two or three smaller incisions may be made to access the hernia site. A laparoscope may be inserted into one incision, and surgical instruments may be inserted into the other incision(s) to remove tissue and place the graft member 80 in the same position as the open procedure.

The graft member 80 may comprise any suitable material for covering the perforation 75 and substantially or entirely inhibiting the protrusion of abdominal matter. In one embodiment, the graft member 80 may comprise small intestinal submucosa (SIS), such as SURGISIS® BIODESIGN™ Soft Tissue Graft, available from Cook Biotech, Inc., West Lafayette, Ind., which provides smart tissue remodeling through its three-dimensional extracellular matrix (ECM) that is colonized by host tissue cells and blood vessels, and provides a scaffold for connective and epithelial tissue growth and differentiation along with the ECM components. Preferably, the graft member 80 would be a one to four layer lyophilized soft tissue graft made from any number of tissue engineered products. Reconstituted or naturally-derived collagenous materials can be used, and such materials that are at least bioresorbable will provide an advantage, with materials that are bioremodelable and promote cellular invasion and ingrowth providing particular advantage. Suitable bioremodelable materials can be provided by collagenous ECMs possessing biotropic properties, including in certain forms angiogenic collagenous extracellular matrix materials. For example, suitable collagenous materials include ECMs such as submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa, including small intestinal submucosa, stomach submucosa, urinary bladder submucosa, and uterine submucosa. The graft member 80 may also comprise a composite of a biomaterial and a biodegradeable polymer. Additional details may be found in U.S. Pat. No. 6,206,931 to Cook et al., the disclosure of which is incorporated herein by reference in its entirety.

Referring now to FIGS. 6-7, after the graft member 80 has been placed to cover the perforation 75, the insertion tool 50 may be advanced to pierce through the graft member 80, and further may pierce at least partially into the tissue 74 at a first location around the perimeter of the perforation 75. Alternatively, in another technique, the insertion tool 50 may be advanced from an opposing direction, i.e., such that it first pierces the tissue 74 and then subsequently pierces through the graft member 80. The principles of the present invention apply regardless of which direction the insertion tool 50 is advanced and the tacking device is deployed.

In this example, the insertion tool 50 is carrying six sequential tacking devices 20a-20f, which may be disposed within the hollow lumen 54 of the insertion tool 50 as shown and explained with respect to FIG. 3 above. With each of the tacking devices 20a-20f in the contracted delivery states, the sharpened tip 52 of the insertion tool 50 may be advanced to a predetermined depth into the tissue 74. The markers 56 of FIGS. 2-3 may facilitate in determining how far the insertion tool 50 has penetrated into tissue 74, as depicted in FIG. 7.

In a next step, the stylet 60 of FIG. 3 may be held steady with respect to the insertion tool 50, while the insertion tool 50 is retracted in a proximal direction. Alternatively, the stylet 50 may be distally advanced, while the insertion tool 50 is held steady, to distally advance the tacking device 20 relative to the insertion tool 50. This causes the distal deployable members 45-47 of the most distal tacking device 20a to extend distal to the sharpened tip 52 of the insertion tool 50, as depicted in FIG. 7. When the distal deployable members 45-47 are no longer radially constrained by the insertion tool 50, they may assume their predetermined expanded configurations in which they may engage, penetrate and/or abut the tissue 74. The deployable members may comprise a spring strength to retroflex inside of a tissue layer to the configuration shown.

As the insertion tool 50 further is retracted proximally with respect to the tacking device 20a, the proximal deployable members 35-37 may assume their predetermined expanded configuration when are no longer radially constrained, as shown in FIG. 7. In the expanded configuration, the proximal deployable members 35-37 may engage, penetrate and/or abut the graft member 80 and optionally penetrate into the tissue 74. In this manner, the tacking device 20a helps secure the graft material 80 against the tissue 74. In particular, the substantially 180-degree hook-shaped configuration of the proximal deployable members 35-37 may urge the graft member 80 in a distal direction towards the tissue 74.

After the first tacking device 20a has been deployed, the insertion tool 50 may be repositioned to deploy another tacking device around the perimeter of the perforation 75. Each subsequent tacking device 20b-20f may be deployed in the same manner as the tacking device 20a. In this manner, the tacking devices 20a-20f may secure the graft member 80 around the perimeter of the perforation 75, as shown in FIG. 9. As will be apparent, greater or fewer tacking devices may be used, and the positioning of the tacking devices may be varied to optimize securing the graft member 80 to the tissue 74 in order to substantially seal the perforation 75.

Optionally, the sheath member 58 of FIG. 2 may be longitudinally advanced over the insertion tool 50, for example, if needed to protect the sharpened distal tip 52 of the insertion tool 50 while the insertion tool 50 is being repositioned. Further, the sheath member 58 may be advanced distally over the insertion tool 50 to facilitate deployment of the proximal deployable members 35-37. For example, the sheath member 58 may periodically push against the graft member 80, thereby temporarily urging the graft member 80 and/or the tissue 74 in a distal direction. At this time, the sheath member 58 may be held steady while the insertion tool 50 is retracted proximally to deploy the proximal deployable members 35-37 at a location proximal to the compressed tissue 74 and graft member 80. Once the proximal deployable members 35-37 have been deployed, the compressive force applied by the sheath member 58 may be removed so that the graft member 80 and the tissue 74 may engage the deployed proximal deployable members 35-37.

In the embodiment of FIGS. 4-9, the tissue 74 illustratively comprises a thickness t₁, while the graft member 80 comprises a thickness t₂. The distal deployable members 45-47 may be deployed entirely within the tissue 74, as depicted in FIG. 8, or alternatively may be deployed substantially distal to the tissue 74 while abutting or piercing through a distal edge of the tissue 74. In the latter embodiment, the longitudinal distance L₁ between the ends 39 and 49 of the tacking device 20 may be dimensioned to be substantially equal to, or slightly less than, the combined thickness t₁+t₂ of the tissue 74 and the graft member 80. The longitudinal distance L₁ may be otherwise sized and configured, as desired, to apply desired forces upon the graft member 80 and the tissue 74.

While FIGS. 4-9 have illustrated the use of one or more tacking device 20 for covering a perforation 75 formed in the ventral abdominal wall, the tacking devices disclosed herein may be useful in many other procedures. Solely by way of example, one or more tacking devices 20 may be used to treat perforations in a visceral wall, such as the stomach wall. In such cases, a suitable insertion device, such as an endoscope, may be advanced through a bodily lumen such as the alimentary canal to a position proximate the target location. One or more components may be advanced through a working lumen of the endoscope. To close the perforation, the graft member 80 may cover the perforation and may be secured in a position overlapping the perforation using the one or more of the tacking devices 20, which may be deployed using the techniques described hereinabove.

Referring now to FIG. 10, in an alternative embodiment, a tacking device 120 may comprise one or more features for facilitating suturing, and preferably purse-string suturing. The tacking device 120 is similar to the tacking device 20 of FIG. 1, except as noted below. The tacking device 120 comprises proximal and distal deployable members 135-137 and 145-147, respectively. In this embodiment, the tacking device 120 comprises a proximal tube portion 122 and distal tube portion 123 with an opening, slot or cutout disposed therebetween, as shown in FIG. 10. First, second and third wires 125-127 may be disposed through the entirety of the proximal and distal tube portions 122 and 123, as depicted in FIG. 10.

The first wire 125 may comprise a proximal end that forms deployable member 135 and a distal end that forms deployable member 145, such that a central region of the first wire 125 is disposed through both tube portions 122 and 123. Similarly, the second and third wires 126 and 127 may be disposed through the entirety of the tube portions 122 and 123. The second wire 126 may comprise a proximal end that forms deployable member 136 and a distal end that forms deployable member 146, while the third wire 127 may comprise a proximal end that forms deployable member 137 and a distal end that forms deployable member 147. The three wires 125-127 may be affixed to an interior surface of the tube portions 122 and 123, for example, using an adhesive, frictional fit or mechanical device. Alternatively, the tube portions 122 and 123 may be omitted, and central regions of the first, second and third wires 125-127 may be affixed to one another, for example, using a solder or weld.

In the embodiment shown, the second wire 126 comprises a loop member 150, which may be formed by bending a central region of the wire that is disposed between the tube portions 122 and 123, as shown in FIG. 10. The second wire 126 may be bent to form an arch-shaped loop member 150 having an aperture 152. A suture 160 may be threaded through the aperture 152 of the loop member 150, for example, as shown in FIG. 11 below.

In alternative embodiments, one single tube member may be employed, in lieu of the proximal and distal tube portions 122 and 123, and the single tube member may comprise a slot or cutout, such that the loop member 150 may extend radially through the slot or cutout. There also may be a single strip of material connecting the proximal and distal tube portions 122 and 123. Further, the loop member 150 need not be formed integrally from any of the wires 125-127, but rather may be formed as a loop disposed on an exterior surface of the proximal and distal tube portions 122 and 123, or on an exterior surface of a single tube member if only one tube is used. Still further, while the loop member 150 is shown in a substantially central location, it may be placed closer to the proximal or distal ends of the tacking device 120.

Referring now to FIG. 11, an exemplary method of using the tacking device 120 is shown. In one step, a graft member 80 may be placed over a perforation 75, and multiple tacking devices 120 may be deployed using an insertion device to secure the graft member 80 to the tissue 74, as explained in detail above with respect to FIGS. 4-9. In the embodiment of FIG. 11, multiple tacking devices 120 may be linked together by a single suture 160, which may be slidably coupled through the loop members 150 of each of the tacking devices 120, as generally shown in FIG. 11. There are two free ends 161 and 162 of the suture 160, which may be independently tensioned to facilitate closure of the perforation 75.

Preferably, multiple tacking devices 120 having loop members 150 are sequentially positioned around the perforation 75 in a semi-annular or annular shape, for example, as shown above in FIG. 9. The ends 161 and 162 of the suture 160 are then tensioned to reduce the distance between the tacking devices and compress the tissue 74 around the perforation 75. The suture ends 161 and 162 may be secured to maintain the compression of the tissue 74 using any suitable technique such as by forming a knot or using clamps, rivets and the like.

Further, in lieu of the loop members 150 described herein, other mechanisms for engaging and/or retaining sutures may be integrally formed with the tacking device 120 or externally attached thereto. Solely by way of example, such suture retaining mechanisms are explained in pending U.S. patent application Ser. No. 11/946,565, filed Nov. 28, 2007, the entire disclosure of which is hereby incorporated by reference in its entirety.

Various types of sutures 160 may be used in conjunction with embodiment of FIGS. 10-11. For example, synthetic sutures may be made from polypropylene, nylon, polyamide, polyethylene, and polyesters such as polyethylene terephthalate. These materials may be used as monofilament suture strands, or as multifilament strands in a braided, twisted or other multifilament construction.

While the examples shown above have illustratively described a tacking device that may be useful for coupling a graft member to tissue to cover and seal a perforation, the tacking devices 20 and 120 also may be used in other procedures. As noted above, the tacking devices 20 and 120 may be used to treat bodily walls during translumenal procedures. Further, the tacking devices 20 and 120 may be used to secure a graft member to tissue for reconstructing local tissue, and the like.

In yet further applications within the scope of the present embodiments, the tacking devices 20 and 120 need not be used for coupling a graft member to tissue. For example, the tacking devices 20 and 120 may be used in an anastomosis procedure. In order to create an anastomosis, for example, multiple tacking devices 20 or 120 may be deployed in a circular manner to couple a proximal vessel, duct or organ to a distal vessel, duct or organ. In such cases, a suitable insertion device, such as an endoscope, may be advanced through a bodily lumen such as the alimentary canal to a position proximate the target location. One or more components, such as the insertion tool 50, may be advanced through a working lumen of the endoscope. The distal end of the insertion tool 50 may be viewed under fluoroscopy, or via optical elements of the endoscope, or by some other visualization technique. Under suitable visualization, multiple tacking devices then may be delivered at one time, for example, using the insertion tool 50. Then, a hole may be punched through the middle of the deployed tacking devices to create a flow path between the proximal and distal vessels/ducts/organs. It will be apparent that still further applications of the tacking devices 20 and 120 are possible. Moreover, the insertion tool 50 may be used with or without an endoscope or similar device.

Referring now to FIG. 12, a further alternative tacking device 220 is similar to the tacking device 20 described above, with a main exception that end regions 231 of the proximal deployable members 35-37 are substantially parallel to the longitudinal axis in the expanded state shown. Specifically, the proximal deployable members 35-37 retroflex in the expanded state such that the end regions 231 are radially spaced apart from, and substantially parallel to, the longitudinal axis of the tacking device. Similarly, end regions 241 of the distal deployable members 45-57 are substantially parallel to the longitudinal axis in the expanded state shown, and therefore, the end regions 231 and 241 of the proximal and distal deployable members are substantially parallel to one another both in the contracted state of FIG. 3 and in the expanded state of FIG. 12. Further, in FIG. 12, the longitudinal distance L₂ between the ends 39′ and 49′ of the tacking device 220 may be less than the longitudinal distance L₁ between the 39 and 49 of the tacking device 20, as depicted in FIG. 1, due to the increased longitudinal length provided by the end regions 231 and 241.

Referring now to FIG. 13, in an alternative tacking device 330, the tube members 22 and 122 shown above have been omitted, and central regions of first, second and third wires 325-327 are affixed to one another, for example, using a solder or weld 329. Notably, the solder or weld 329 holds the central regions of the first, second and third wires 325-327 together, but does not interfere with expansion of the proximal and distal deployable members 35-37 and 45-47, as described above.

Referring now to FIGS. 14-15, in further alternative embodiment, one or more proximal or distal deployable members may comprise a rounded curvature that may engage a graft member or tissue in a less traumatic manner. For example, in FIG. 14, an alternative proximal deployable member 37′ spans about 270 degrees, such that the end 39 is substantially parallel to the tissue or graft member, as opposed to substantially perpendicular. In FIG. 15, a further alternative proximal deployable member 37″ spans greater than 360 degrees, and a rounded region 331 may engage the tissue or graft member in a less traumatic manner. It will be appreciated that while only the proximal deployable member 37 is shown having a modified, curved design, any or all of the proximal and distal deployable members 35-37 and 47-47 may comprises the curvatures shown.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

1-20. (canceled)

21. A tacking device comprising:
a first wire having proximal and distal ends, a proximal deployable member formed at the proximal end of the first wire, and a distal deployable member formed at the distal end of the first wire;
a second wire having proximal and distal ends, a proximal deployable member formed at the proximal end of the second wire, and a distal deployable member formed at the distal end of the second wire;
wherein the proximal deployable members of the first and second wires have contracted and expanded states, and further wherein the distal deployable members of the first and second wires have contracted and expanded states; and
at least one tube member having a proximal end and a distal end,
wherein the first and second wires are disposed through the tube member, and
wherein the proximal deployable members of the first and second wires extend proximally beyond the proximal end of the tube member, and wherein the distal deployable members of the first and second wires extend distally beyond the distal end of the tube member.

22. The tacking device of claim 21 further comprising a third wire having proximal and distal ends, a proximal deployable member formed at the proximal end of the third wire, and a distal deployable member formed at the distal end of the third wire,
wherein the proximal deployable member of the third wire has contracted and expanded states, and further wherein the distal deployable member of the third wire has contracted and expanded states,
wherein each of the first, second and third wires is disposed through the tube member, and
wherein each of the proximal deployable members of the first, second and third wires extends proximally beyond the proximal end of the tube member, and wherein each of the distal deployable members of the first, second and third wires extends distally beyond the distal end of the tube member.

23. The tacking device of claim 22, wherein the first, second and third wires are affixed to an interior surface of the tube member.

24. The tacking device of claim 22, wherein the first, second and third wires are sized to create a frictional fit against each other and an interior surface of the tube member, thereby inhibiting movement with respect to the tube member.

25. The tacking device of claim 21, wherein an end region of the distal deployable member of the first wire is substantially parallel to a longitudinal axis of the first wire in the contracted state, and further wherein the distal deployable member of the first wire retroflexes in the expanded state to be radially spaced apart from, and substantially parallel to, the longitudinal axis of the first wire.

26. The tacking device of claim 25, wherein an end region of the proximal deployable member of the first wire is substantially parallel to a longitudinal axis of the first wire in the contracted state, and further wherein the proximal deployable member of the first wire retroflexes in the expanded state to be radially spaced apart from, and substantially parallel to, the longitudinal axis of the first wire.

27. The tacking device of claim 26, wherein a longitudinal distance between the end region of the distal deployable member of the first wire and the end region of the proximal deployable member of the first wire is dimensioned to be substantially equal to or less than the combined thickness and of a tissue and a graft member to provide a desired compressive force upon the tissue and the graft member.

28. The tacking device of claim 21, wherein the proximal and distal deployable members of the first and second wires comprise hook-shaped configurations in the expanded states.

29. A tacking device comprising:
a plurality of proximal deployable members, each having proximal and distal ends and having contracted and expanded states; and
a plurality of distal deployable members, each having proximal and distal ends and having contracted and expanded states; and
at least one tube member having a proximal end and a distal end,
wherein the plurality of proximal deployable members are coupled to the proximal end of the tube member,
wherein the plurality of distal deployable members are coupled to the distal end of the tube member, and
wherein the plurality of proximal deployable members extend proximally beyond the proximal end of the tube member, and wherein the plurality of distal deployable members extend distally beyond the distal end of the tube member.

30. The tacking device of claim 29, wherein each of the plurality of proximal deployable members is affixed to an interior surface of the tube member.

31. The tacking device of claim 29, wherein each of the plurality of proximal deployable members is sized to create a frictional fit against each other and an interior surface of the tube member, thereby inhibiting movement with respect to the tube member.

32. The tacking device of claim 29, wherein each of the plurality of distal deployable members is affixed to an interior surface of the tube member.

33. The tacking device of claim 29, wherein each of the plurality of distal deployable members is sized to create a frictional fit against each other and an interior surface of the tube member, thereby inhibiting movement with respect to the tube member.

34. The tacking device of claim 29, wherein an end region of each of the distal deployable members is substantially parallel to a longitudinal axis of the tube member in the contracted state, and further wherein each of the distal deployable members retroflexes in the expanded state to be radially spaced apart from, and substantially parallel to, the longitudinal axis of the tube member.

35. The tacking device of claim 34, wherein an end region of each of the proximal deployable members is substantially parallel to a longitudinal axis of the tube member in the contracted state, and further wherein each of the proximal deployable members retroflexes in the expanded state to be radially spaced apart from, and substantially parallel to, the longitudinal axis of the tube member.

36. The tacking device of claim 29, wherein each of the proximal and distal deployable members comprises hook-shaped configurations in the expanded states.
